(19) United States
(12) Patent Application Publication
(10) Pub. No.: US 2010/0010309 A1
(43) Pub. Date: Jan. 14, 2010

(54) ENDOSCOPE BEND TUBE AND ENDOSCOPE INCLUDING THE BEND TUBE

(75) Inventor: Hideya KITAGAWA, Hachioji-shi (JP)

Correspondence Address: Frishauf, Holtz Goodman & Chick, P.C., 220 Fifth Avenue - 16th Floor, New York, NY, 10001-7708, US

(73) Assignee: OLYMPUS CORPORATION, Tokyo (JP)

(21) Appl. No.: 12/486,517

(22) Filed: Jun. 17, 2009

(30) Foreign Application Priority Data
Jul. 14, 2008 (JP) 2008-183168

(51) Int. Cl. A61B 1/008

(52) U.S. Cl. 600/142

(57) ABSTRACT

An endoscope bend tube includes a plurality of node rings, wherein contact portions at which neighboring ones of the node rings are pit in contact are provided between the neighboring node rings, and the plurality of node rings are coupled to be pivotable about pivotal center axes which correspond to pivotal centers of the contact portions, the endoscope bend tube includes an operation wire, a coupling wire, and a coupling wire hold portion, wherein in a case where the endoscope bend tube is in a non bent state, lead-out ends of the coupling wire hold portions, which are opposed between a pair of the neighboring node rings, are disposed at such positions that a line segment connecting the lead-out ends is halved by a plane including a center axis in a longitudinal direction of the bend tube and a pivotal center axis of the pair of the node rings.

### CROSS-REFERENCE TO RELATED APPLICATIONS

This application is based upon and claims the benefit of priority from prior Japanese Patent Application No. 2008-183168, filed Jul. 14, 2008, the entire contents of which are incorporated herein by reference.

### BACKGROUND OF THE INVENTION

1. Field of the Invention

The present invention relates to an endoscope bend tube, and an endoscope including the bend tube.

2. Description of the Related Art

In general, an insertion section of an endoscope is provided with a bend section. A skeleton structure of the bend section is a bend tube which is configured such that a plurality of bend pieces (node rings) are arranged in line in the major axis direction (longitudinal direction) of the bend section, and bend pieces, which neighbor in the back-and-forth direction, are pivotably coupled. Most of conventional bend tubes are configured such that the bend pieces, which neighbor in the back-and-forth direction, are pivotably coupled by pivotal support pins. Specifically, tongue portions are formed at end edges of each bend piece. A tongue portion of one bend piece is laid over a tongue portion of a neighboring bend piece. Further, a rivet-like pivotal support pin is attached so as to penetrate both tongue portions that are laid over each other. The front and rear bend pieces are configured to be pivotably coupled by the rivet-like pivotal support pin.

Jpn. Pat. Appln. KOKAI Publication No. 8-129131 (patent document 1) proposes a bend tube having the following structure, without a rivet-like pivotal support pin. In this structure, neighboring bend pieces are partly put in contact with each other, and the bend pieces are covered with an outer sheath and are coupled in a manner to be pivotable about a specified axis. In this contact-type bend tube, coupling means is needed to specify the entire length in the major axis direction of the bend tube, thereby to keep the contact state of the bend pieces. In the above-described patent document 1, the entire length in the major axis direction of the bend tube is specified by making use of the bending characteristics at a close contact time of a net-like tube of the outer sheath.

Jpn. Pat. Appln. KOKAI Publication No. 2005-230182 (patent document 2) discloses a different coupling structure of a bend tube which is configured such that neighboring bend pieces are partly put in contact and are coupled to be pivotable about a specified axis. In this structure, coupling wires, which are different from operation wires of a bend section, are provided. The coupling wires are disposed near contacting pivotal portions of coupling structure parts so as to penetrate straight in the major axis direction of the bend tube. The contacting pivotal coupling portions are fixed by the coupling wires so that they are not detached.

### BRIEF SUMMARY OF THE INVENTION

According to an aspect of the present invention, there is provided an endoscope bend tube including a plurality of node rings which are arranged in line, wherein contact portions at which neighboring ones of the node rings are put in contact are provided between the neighboring node rings, and the plurality of node rings are coupled to be pivotable about pivotal center axes which correspond to pivotal centers of the contact portions, the endoscope bend tube comprising: an operation wire which is disposed in the bend tube and bends the bend tube; a coupling wire which is disposed between the node rings and couples the node rings; and a coupling wire hold portion which is provided in the node ring and holds the coupling wire, wherein in a case where the endoscope bend tube is in a non-bent state, lead-out ends of the coupling wire hold portions, which are opposed between a pair of the neighboring node rings, are disposed at such positions that a line segment connecting the lead-out ends is halved by a plane including a center axis in a longitudinal direction of the bend tube and a pivotal center axis of the pair of the node rings.

Preferably, the lead-out end of the coupling wire hold portion of one of the paired node rings and the lead-out end of the coupling wire hold portion of the other node ring that neighbors and is opposed to the one of the paired node rings are disposed at positions of two-fold rotational symmetry about the center axis in the longitudinal direction of the bend tube.

Preferably, the lead-out end of the coupling wire hold portion of one of the paired node rings and the lead-out end of the coupling wire hold portion of the other node ring that is opposed to the one of the paired node rings are disposed at positions of four-fold rotational symmetry about the center axis in the longitudinal direction of the bend tube.

Preferably, the coupling wire is disposed in a path passing through the line segment.

Preferably, each of the node rings includes at least two the coupling wire hold portions, and the node rings are coupled by two the coupling wires which are passed through the coupling wire hold portions of each node ring.

Preferably, each of the node rings includes at least four the coupling wire hold portions, and the node rings are coupled by four the coupling wires which are passed through the coupling wire hold portions or each node ring.

Preferably, the node ring includes an operation wire hold portion which holds the operation wire which bends the endoscope bend tube, and the coupling wire hold portion is disposed between the operation wire hold portion and a position corresponding to a part where the contact portion at which the neighboring node rings are put in contact is provided.

Preferably, the endoscope bend tube includes two the operation wires, and the endoscope bend tube is bendable in two directions by the two operation wires.

Preferably, the endoscope bend tube includes four the operation wires, and the endoscope bend tube is bendable in four directions by the four operation wires.

Preferably, the coupling wire is a metallic stranded wire.

Preferably, the coupling wire is a resin twisted thread.

According to another aspect of the present invention, there is provided an endoscope including an endoscope bend tube which includes a plurality of node rings which are arranged in line, wherein contact portions at which neighboring ones of the node rings are put in contact are provided between the neighboring node rings, and the plurality of node rings are coupled to be pivotable about pivotal center axes which correspond to pivotal centers of the contact portions, the endoscope comprising: an operation wire which is disposed in the bend tube and bends the bend tube; a coupling wire which is disposed between the node rings and couples the node rings; and a coupling wire hold portion which is provided in the node ring and holds the coupling wire, wherein in a case where the endoscope bend tube is in a non-bent state, lead-out ends of the coupling wire hold portions, which are opposed between a pair of the neighboring node rings, are disposed at such positions that a line segment connecting the lead-out ends is halved by a plane including a center axis in a longitudinal direction of the bend tube and a pivotal center axis of the pair of the node rings.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view of an endoscope including a bend tube according to a first embodiment of the present invention;

FIG. 2A is a side view of the bend tube according to the first embodiment;

FIG. 2B is a rear view which shows the bend tube as viewed from the rear side thereof;

FIG. 2C is a bottom view of the bend tube;

FIG. 3 is a perspective view of one of node rings which constitute the bend tube of the first embodiment;

FIG. 4 is a perspective view of the bend tube in a state which the node rings of the first embodiment are coupled;

FIG. 5A is a side view showing terminal end portions of distal ends of coupling wires of the bend tube of the first embodiment;

FIG. 5B is a side view showing terminal end portions of proximal ends of coupling wires of the bend tube of the first embodiment;

FIG. 6A is an explanatory view showing the arrangement of the coupling wires which couple the node rings in the bend tube of the first embodiment;

FIG. 6B is an explanatory view showing the arrangement of the coupling wires which couple the node rings in the bend tube in a case where the bend tube of the first embodiment is bent;

FIG. 7 is a table showing the variation of an inter-opening distance (L) of coupling wire hold portions of neighboring node rings, relative to a bend angle (θ) of the neighboring node rings;

FIG. 8 is a graph showing the relationship between the bend angle (θ) of the neighboring node rings and the inter-opening distance (L) of the coupling wire hold portions;

FIG. 9A is a side view showing an endoscope bend tube according to a second embodiment of the present invention;

FIG. 9B is a rear view which shows the bend tube as viewed from the rear side thereof;

FIG. 9C is a bottom view of the bend tube;

FIG. 10 is a perspective view of one of node rings which constitute the endoscope bend tube of the second embodiment;

FIG. 11 is a perspective view of the bend tube in a state which the node rings of the second embodiment are coupled;

FIG. 12A is a side view showing an endoscope bend tube according to a third embodiment of the present invention;

FIG. 12B is a rear view which shows the bend tube as viewed from the rear side thereof;

FIG. 12C is a bottom view of the bend tube;

FIG. 13 is a perspective view of one of node rings which constitute the endoscope bend tube of the third embodiment;

FIG. 14 is a perspective view of the bend tube in a state which the node rings of the third embodiment are coupled;

FIG. 15A is a side view showing an endoscope bend tube according to a fourth embodiment of the present invention;

FIG. 15B is a rear view which shows the bend tube as viewed from the rear side thereof;

FIG. 15C is a bottom view of the bend tube;

FIG. 16 is a perspective view of one of node rings which constitute the endoscope bend tube of the fourth embodiment;

FIG. 17 is a perspective view of the bend tube in a state which the node rings of the fourth embodiment are coupled;

FIG. 18A is a side view showing an endoscope bend tube according to a fifth embodiment of the present invention;

FIG. 18B is a rear view which shows the bend tube as viewed from the rear side thereof;

FIG. 19 is a perspective view of one of node rings which constitute the endoscope bend tube of the fifth embodiment;

FIG. 20A is a side view showing an endoscope bend tube according to a sixth embodiment of the present invents on;

FIG. 20B is a rear view which shows the bend tube as viewed from the rear side thereof; and

FIG. 21 is a perspective view of one of node rings which constitute the endoscope bend tube of the sixth embodiment.

### DETAILED DESCRIPTION OF THE INVENTION

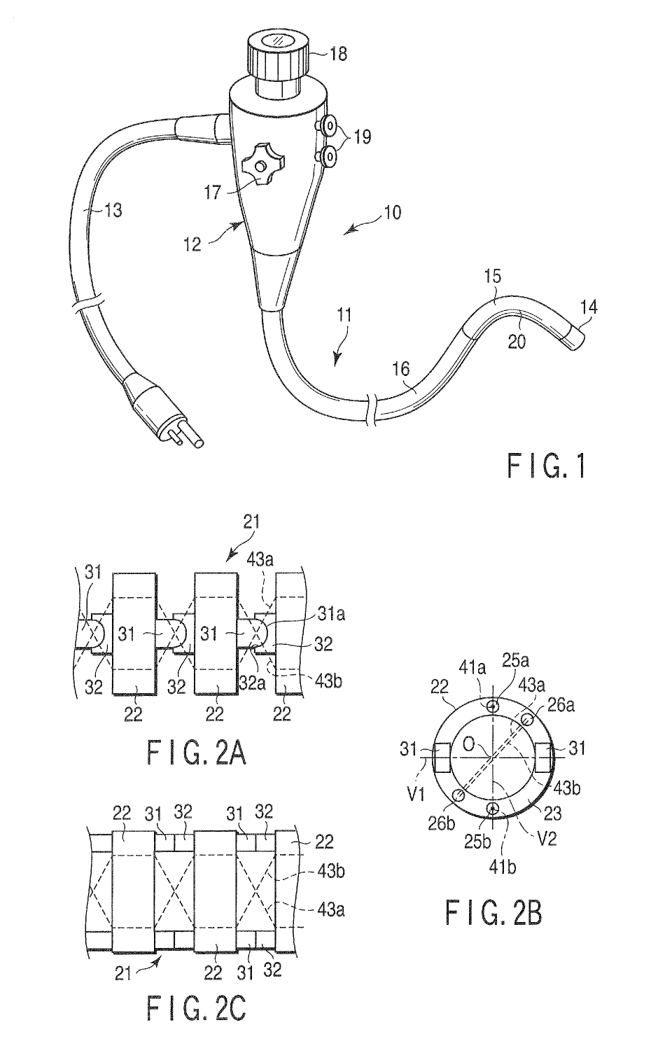

Embodiments of the present invention will now be described with reference to the accompanying drawings. FIG. 1 to FIG. 8 show an endoscope bend tube according to a first embodiment of the present invention, and an endoscope including this bend tube.

As is shown in FIG. 1, an endoscope 10 according to this embodiment includes an elongated insertion section 11 which is inserted into a subject body, an operation section 12 serving also as a handle section, which is coupled to a proximal end portion of the insertion section 11, and a universal cord 13 which is connected to the operation section 12. The insertion section 11 is formed by coupling a distal-end structure section 14, a bend section 15 and an elongated flexible tube section 16. The distal-end structure section 14, bend section 15 and flexible tube section 16 are disposed in the named order from the distal end side of the insertion section 11. The operation section 12 is provided with an operation knob 17 for bend-operating the bend section 15, an eyepiece section 18, and operation buttons 19 for air/water feed.

The bend section 15 includes a bend tube functioning as a skeleton structure (core member) that is to be described later. The bend tube is covered with an outer sheath 20 serving as an outer cover member, which is shown in FIG. 1. The outer sheath 20 may be composed of a braid tube and a rubber outer sheath. Alternatively, the outer sheath 20 may be formed of a rubber outer sheath alone, or a single-layer outer sheath.

FIG. 2A, FIG. 2C and FIG. 4 show the structure of a bend tube 21 functioning as the skeleton structure of the bend section 15. A plurality of node rings (pieces) 22 are arranged in line in the longitudinal direction of the bend section 15 (the direction of a center axis (O)). Node rings 22, which neighbor in the back-and-forth direction, are coupled via swivel contact portions so as to be vertically pivotable about pivotal centers of the swivel contact portions.

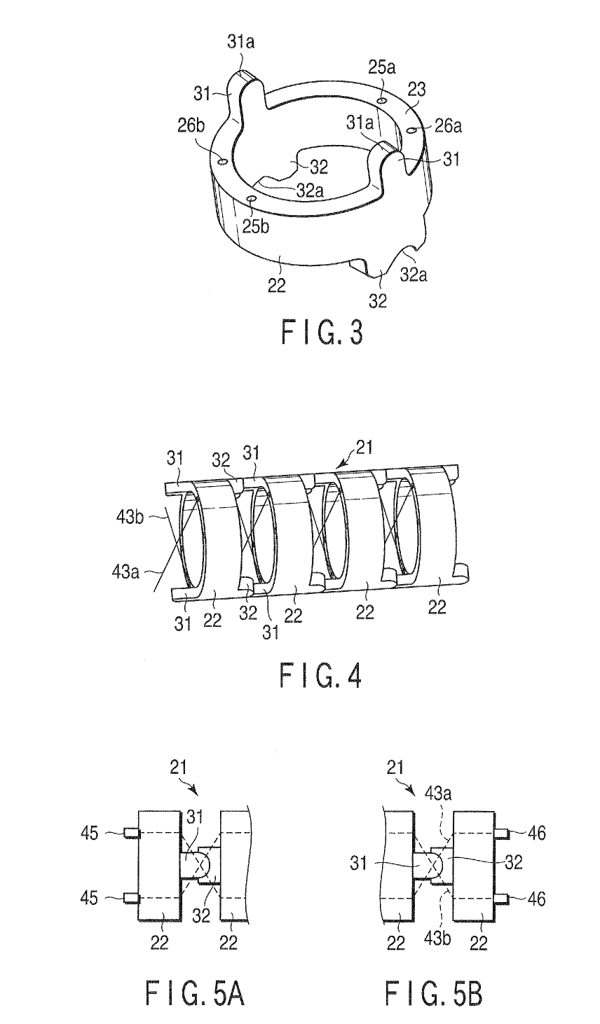

The foremost node ring 22 is connected to a member of the distal-end structure section 14. The rearmost node ring 22 is connected to a distal end of the flexible tube section 16, directly or via a member such as a connection tube. Since the node ring 22, which is an element of the bend tube 21, is formed to be a short, tubular member as a single unit as shown in FIG. 3, this tubular member is called “node ring”. However, the node ring 22 is not necessarily limited to the tubular member.

FIG. 3 shows the node ring 22 as a single unit. The single-unit node ring 22 includes a short tubular node ring body 23. A pair of tongue-shaped protrusion portions 31 are formed on one of end edges of the node ring body 23. The protrusion portions 31 are disposed at positions of 180° in the circumferential direction of the node ring body 23. A pair of tongue-shaped reception portions 32 are formed on the other end edge of the node ring body 23. The reception portions 32 are similarly disposed at positions of 180° in the circumferential direction of the node ring body 23. Arcuate projection portions 31a are formed at projecting distal ends of the protrusion portions 31. Arcuate recess portions 32a are formed at projecting distal ends of the reception portions 32. The projection portion 31a of the protrusion portion 31 and the recess portion 32a of the reception portion 32 have the same size (radius) of an arc.

As shown in FIG. 2B, in the node ring body 23, an imaginary line V1, which passes through the centers of curvature of the arcuate projection portions 31a of the paired protrusion portions 31, is disposed so as to pass through the center axis (O) of the node ring body 23 (i.e. so as to intersect with the center axis (O)). Thereby, the paired protrusion portions 31 are disposed to be symmetric in the right-and-left direction with respect to a line V2 which is perpendicular to the imaginary line V1. Similarly, in the node ring body 23, the paired reception portions 32 are disposed to be symmetric in the right-and-left direction at the other end edge of the node ring body 23. Thereby, an imaginary line V1, which passes through the centers of curvature of the arcuate recess portions 32a at the projecting distal ends of the paired left and right reception portions 32, is disposed so as to pass through the center axis (O) of the node ring body 23 (i.e. so as to intersect with the center axis (O)). The protrusion portions 31 of one of the node rings 22, which neighbor in the back-and-forth direction, are put in contact with the reception portions 32 of the other node ring 22. Thus, the paired node rings 22, which neighbor in the back-and-forth direction, constitute swivel contact portions which are coupled to be vertically pivotable about pivotal centers that are the centers of curvature of the arc.

The tongue-shaped protrusion portions 31 and tongue-shaped reception portions 32 are formed integral with the node ring body 23. The node ring 22 is formed of a hard material, for example, a metallic material such as stainless steel or aluminum, by molding or cutting, and the surface of the node ring 22 is subjected to a lubricating surface treatment process. Alternatively, the entire node ring 22 may be integrally formed of a resin material, such as a high-hardness resin, by injection molding.

As shown in FIG. 2B, the node ring body 23 is provided with operation wire hold portions which hold operation wires. In FIG. 2B, the operation wire hold portions are formed of operation wire guide holes 25a and 25b which are provided at upper and lower positions of the node ring body 23. The operation wire guide holes 25a and 25b are formed so as to penetrate straight in the same direction as the direction of the center axis (O) of the node ring body 23 between an end edge on one side surface of the node ring body 23 and an end edge on the other side surface of the node ring body 23.

In addition, a pair of coupling wire hold holes 26a and 26b, which serve as coupling wire hold portions, are formed in the node ring body 23. The paired coupling wire hold holes 26a and 26b are disposed at positions deviating from the parts of the operation wire guide holes 25a and 25b, protrusion portions 31 and reception portions 32 (i.e. oblique positions at 45° to the up-and-down direction and right-and-left direction in FIG. 2B).

The coupling wire hold holes 26a and 26b are formed so as to penetrate straight in the same direction as the direction of the center axis (O) of the node ring body 23 between the end edge on one side surface of the node ring body 23 and the end edge on the other side surface of the node ring body 23. As shown in FIG. 2B, the paired coupling wire hold holes 26a and 26b are disposed at equidistant positions from the center axis (O) of the node ring body 23. An imaginary line passing through the positions of the paired coupling wire hold portions 26a and 26b is disposed to be symmetric with respect to the center axis (O) so as to pass through the center axis (O) (i.e. so as to cross the center axis (O)).

The paired coupling wire hold holes 26a and 26b are disposed to be two-fold rotational-symmetric with respect to the center axis (O) of the node ring body 23. As shown in FIG. 2B, when the node ring body 23 is viewed in the direction of the center axis (O), the paired coupling wire hold holes 26a and 26b are disposed at an obliquely upper right position and an obliquely lower left position, respectively.

In the present embodiment, the paired operation wire guide holes 25a and 25b, the paired protrusion portions 31, the paired reception portions 32 and the paired coupling wire hold holes 26a and 26b are disposed to be point-symmetric about the center axis (O) of the node ring body 23, respectively. The paired coupling wire hold holes 26a and 26b are disposed at positions deviating from the paired operation wire guide holes 25a and 25h, the paired protrusion portions 31 and the paired reception portions 32 so as to avoid interference with them. The paired coupling wire hold holes 26a and 26b are disposed at oblique positions with displacements of 45° about the center axis of the bend section 15 from the paired operation wire guide holes 25a and 25b, the paired protrusion portions 31 and the paired reception portions 32.

Coupling wires 43a and 43b are passed through the coupling wire hold holes 26a and 26b of each node ring 22 in the following manner. Specifically, one coupling wire 43a is passed through one coupling wire hold hole 26a of one node ring 22, and the other coupling wire 43b is passed through the other coupling wire hold hole 26b. Then, the one coupling wire 43a is passed through the other coupling wire hold hole 26b of the neighboring other node ring 22. Similarly, the other coupling wire 43b is passed through the one coupling wire hold hole 26a of the neighboring other node ring 22. Subsequently, in a similar manner, the two coupling wires 43a and 43b are passed in a so-called cross-coupling fashion such that the two coupling wires 43a and 43b are alternately passed between one coupling wire hold hole 26a of one of neighboring node rings 22 and the other coupling wire hold hole 26b of the other node ring 22, and between the other coupling wire hold hole 26b of the one of the neighboring node rings 22 and the one coupling wire hold hole 26a of the other node ring 22. Thereby, the plural node rings 22, which are arranged in line, are coupled.

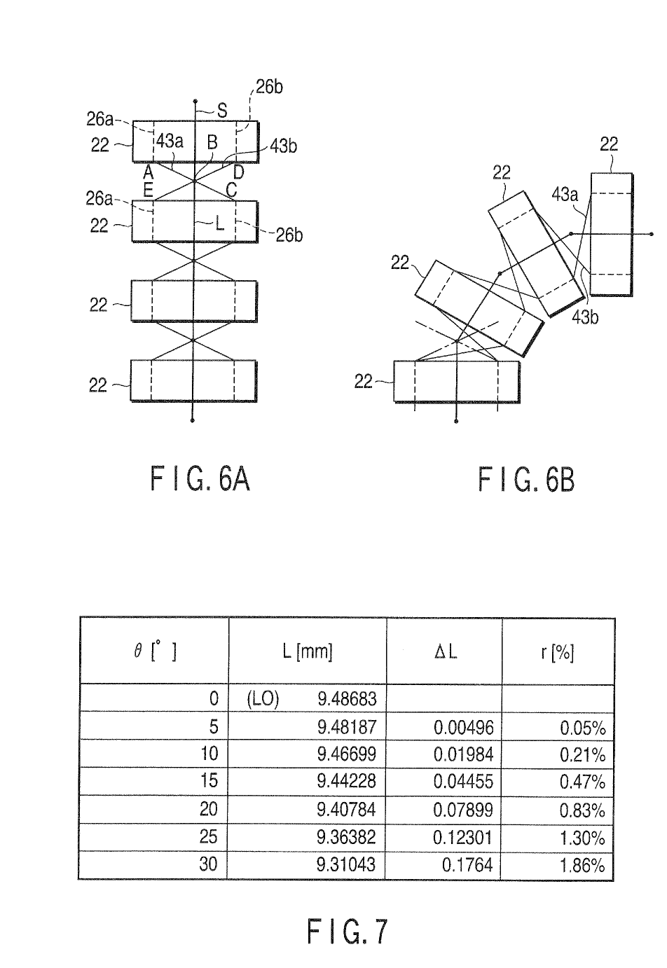

As shown in FIG. 6A, assume now that a lead-out end of the coupling wire hold hole 26a, which is positioned on the upper side of one of a pair of node rings 22 which neighbor and are opposed to each other, is A, and a lead-out end of a coupling wire hold hole 26b, which is positioned on the lower side of the other node ring 22, is C. At this time, a plane including a bend section axis (agreeing with the center axis (O) of the node ring body 23), which is the center axis (O) in the longitudinal direction of the bend tube 21 in the state in which the bend tube 21 is not bent and is straight (i.e. the nor-bent state), intersects with a point (B) at which a line segment “AC” connecting the lead-out ends A and C is halved.

In addition, the line segment “AC” is halved by a plane S including both the center axis (O) in the longitudinal direction of the bend tube 21 and the pivotal center axis of the pair of neighboring node rings 22 (i.e. the rotational center axis in the case where the neighboring node rings 22 are pivoted by the swivel contact portions).

As shown in FIG. 2A, the projection portion 31a of the protrusion portion 31 of the node ring 22, which is positioned on the front side, is engaged in the recess portion 32a of the reception portion 32 of the neighboring node ring 22 which is positioned on the rear side, and the projection portion 31a and the recess portion 32a are engaged in a slidable contact state. Thereby, swivel contact portions are constituted, which become hinge portions of the contact pair for coupling the neighboring front and rear node rings 22 in a manner to be pivotable about the center of the arc of the projection portion 31a and the recess portion 32a.

The center of the arc of the swivel contact portion constitutes the pivotal center axis in the case where the paired neighboring node rings 22 are pivoted. Thereby, the paired neighboring node rings 22, which neighbor at the swivel contact portion, are coupled to be vertically pivotable in FIG. 2A. Thus, in the bend tube 21, the paired neighboring node rings 22 are vertically pivotable, and the entire bend section 15 are vertically bendable. The protrusion portion 31 and reception portion 32, which constitute the swivel contact portion, protrude from the end edges of the node ring body 23. Accordingly, when the node rings 22, which neighbor in the back-and-forth direction, are pivoted, the node rings 22 neighboring in the back-and-forth direction do not immediately abut upon each other, and an amount of bending of the bend tube 21 is secured.

As shown in FIG. 2B, one bend operation wire 41a is passed through one operation wire guide hole 25a in each of the plural node rings 22, which are arranged in line, and the other bend operation wire 41b is passed through the other operation wire guide hole 25b. Thereby, the one bend operation wire 41a is passed through each of the operation wire guide holes 25a which are positioned on the upper side of the plural node rings 22 which are arranged in line. Similarly, the other bend operation wire 41b is passed through each of the operation wire guide holes 25b which are positioned on the lower side of the plural node rings 22. Distal ends of the bend operation wires 41a and 41b are connected to the foremost node ring 22 of the bend tube 21. Rear ends of the bend operation wires 41a and 41b are led to the operation section 12 through the flexible tube section 16 of the insertion section 11, and are coupled to a bend driving mechanism (not shown) which is assembled in the operation section 12.

When the bend section 15 is in the non-bent state, the two bend operation wires 41a and 41b are disposed at upper and lower positions of the bend tube 21 in the state in which the bend operation wires 41a and 41b are parallel to the center axis (O) in the longitudinal direction of the bend tube 21.

When the operation knob 17 is operated, the bend driving mechanism is driver by the operation of the operation knob 17, thereby pulling one of the upper and lower bend operation wires 41a and 41b. The bend section 15 is bent in the direction of one of the upper and lower bend operation wires 41a and 41b, which is pulled at this time.

Each of the coupling wires 43a and 43, which are passed through the coupling wire hold holes 26a and 16b of each node ring 22, is formed of a metallic wire, for instance, a stranded stainless-steel wire. Alternatively, the coupling wire 43a, 43b may be formed of a resin twisted thread such as a polyethylene twisted thread.

As shown in FIG. 2A, FIG. 2B and FIG. 2C, the two coupling wires 43a and 43b are successively passed in a cross-coupling fashion through the coupling wire hold holes 26a and 26b of the respective node rings 22, with their insertion ends being alternated. Specifically, one coupling wire 43a (or 43b) is passed through the coupling wire hold holes 26a and 26b at different positions of the node rings 22 which neighbor in the back-and-forth direction. For example, the coupling wire 43a, which has been passed through the coupling wire hold hole 26a at the obliquely upper right position of the rear-side node ring 22, is led out of the front-side opening (lead-out end) of the coupling wire hold hole 26a, and then inserted into the coupling wire hold hole 26b at the obliquely lower left position of the front-side node ring 22 from the rear-side opening (lead-out end) of the coupling wire hold hole 26b. Accordingly, the coupling wire 43a is always disposed oblique between the neighboring node rings 22.

The other coupling wire 43b extends obliquely from the front-side opening of the coupling wire hold hole 26b at the obliquely lower left position D of the rear-side node ring 22 toward the coupling wire hold hole 26a at the obliquely upper right position of the front-side neighboring node ring 22. The coupling wire 43b is then inserted into the coupling wire hold hole 26a from the rear-side opening (lead-out end) of the coupling wire hold hole 26a.

The coupling wires 43a and 43b extend obliquely between the node rings 22 which neighbor in the back-and-forth direction, and also the coupling wires 43a and 43b cross each other between these neighboring node rings 22. In short, as shown in FIG. 2A, FIG. 2B and FIG. 2C, the two coupling wires 43a and 43b are alternately passed through the different coupling wire hold holes 26a and 26b of each node ring 22, and are disposed crosswise between the neighboring node rings. As shown in FIG. 2B, the paths of the two coupling wires 43a and 43b between the neighboring node rings 22 are oblique to the plane including both the center axis (O) in the longitudinal direction of the bend tube 21 and the pivotal center axis of pivotal movement of the neighboring paired node rings 22. The angle of the oblique paths to this plane is 45′.

As shown in FIG. 5A, distal-end tips 45 are securely attached by, e.g. caulking, to the distal ends of the coupling wires 43a and 43b. The distal-end tip 45 is formed to have such a diameter that the distal-end tip 45 does not enter the distal-end opening of the coupling wire hold hole 26a, 26b, of the node ring 22. Thus, when a force is produced by some cause to pull the distal end of the coupling wire 43a, 43b into the coupling wire hold hole 26a, 26b, the distal-end tip 45 abuts and hooks on the distal-end surface of the foremost node ring 22. Therefore, the distal end of the coupling wire 43a, 43b does not enter the coupling wire hold hole 26a, 26b.

In addition, as shown in FIG. 3B, rear-end tips 46 are securely attached by, e.g. caulking, to the rear ends of the coupling wires 43a and 43b. The rear-end tip 46 is formed to have such a diameter that the rear-end tip 46 does not enter the rear-end opening of the coupling wire hold hole 26a, 26b of the node ring 22. Thus, when a force is produced by some cause to draw the rear end of the coupling wire 43a, 43b, the rear-end tip 46 does not enter the coupling wire hold hole 26a, 26b and abuts and hocks on the rear-end surface of the rearmost node ring 22. Therefore, the rear end of the coupling wire 43a, 43b does not enter the coupling wire hold hole 26a, 26b.

The length of the coupling wire 43a, 43b is set at such a natural length as to keep the engagement state in which the protrusion portion 31 and the reception portion 32 of the swivel contact portion abut upon each other. At this time, no initial tensile force is applied to the coupling wire 43a, 43b. As described above, the movement of the distal end and rear end of the coupling wire 43a, 43b in the direction of pulling into the bend tube 21 is restricted at the positions of the distal end and rear end of the bend tube 21. Accordingly, the length of the coupling wire 43a, 43b is determined in the state in which the bend section 15 is not bent.

The distal end or rear end of the coupling wire 43a, 43b may be coupled to the foremost node ring 22 or rearmost node ring 72 of the bend tube 21 by adhesion or other fixing means. The distal end of the coupling wire 43a, 43b may be coupled to the member of the distal-end structure section 14. The rear end of the coupling wire 43a, 43b may be connected to the flexible tube section 16, directly or via a connection tube.

Next, a description is given of the operation of the coupling wires 43a and 43b which couple the node rings 22 of the bend tube 21, in the case where the bend section 15 is bent.

FIG. 6A shows the state in which the bend tube 21 is kept in a non-bent state. FIG. 6A shows, in a simplified manner, the state in which two coupling wires 43a and 43b are passed through the coupling wire hold holes 26a and 26b of the rode rings 22. As shown in FIG. 6A, assume now that a coupling wire hold position at the front-si de opening (lead-out end) of a coupling wire hold hole 26a of one (upper-side) node ring 22 of a pair of neighboring node rings 22, through which one coupling wire 43a is passed, is “A”, and a coupling wire hold position at the rear-side opening (lead-out end) of a coupling wire hold hole 26b of the other (lower-side) node ring 22 is “C”. At this time, assume that a line segment (path) connecting the coupling wire hold position “A” and coupling wire hold position “C” is “AC”. In addition, assume that a point at which the line segment “AC” is halved in the straight, non-bent state of the bent tube 21, as shown in FIG. 6A, is “B”.

In the non-bent state of the bend tube 21, when the bend tube 21 is viewed from the lateral side, the point “B” intersects with a plane “S” including the center axis (O) (bend section axis) in the longitudinal direction of the bend tube 21. In short, the point “B” is a point of intersection between the line segment “AC” and the plane “S”. The line segment “AC” is also halved by a plane including the pivotal center axis of the neighboring pair of node rings 22. The point “B”, at which the line segment “AC” is halved is set in such a relationship that the point “B” intersects with the plane “S” including the longitudinal center axis (O) of the bend tube 21 and the pivotal center axis of the paired front and rear node rings 22. The paths of the two coupling wires 43a and 43b cross obliquely, as shown in FIG. 2B, between the neighboring front and rear node rings 22.

On the other hand, as shown it FIG. 6A, assume that a coupling wire hold position at the front-side opening (lead-out end) of a coupling wire hold hole 26b of the one (upper-side) node ring 22 is “D”, and a coupling wire hold position at the rear-side opening 1′ (lead-out end) of a coupling wire hold hole 26a of the other (lower-side) node ring 22 is “E”. A line segment “DE” connecting the coupling wire hold position “D” and coupling wire hold position “E” is, as shown in FIG. 6A, halved by the plane including the pivotal center axis of the neighboring pair of node rings 22 in the non-bent state of the bend tube 21. The point “B”, at which the line segment “DE” is halved, is also a point of intersection with the plane “S” including the longitudinal center axis (O) of the bend tube 21 and the pivotal center axis of the neighboring pair of front and rear node rings 22.

As described above, the coupling wires 43a and 43b are alternately passed through the coupling wire hold holes 26a and 26b of the node rings 22. Accordingly, the pair of coupling wires 43a and 43b extend along crossed paths between the neighboring node rings 22. When the bend tube 21 is bent, as shown in FIG. 6B, each coupling wire 43a, 43b is alternately disposed from the inside of bend (inner periphery of bend) to the outside of bend (outer periphery of bend), and from the outside of bend (outer periphery of bend) to the inside of bend (inner periphery of bend). Thus, the path lengths of the coupling wires 43a and 43b are substantially constant between the node rings 22. Accordingly, as shown in FIG. 6B, even if the bend tube 21 is bent, the path lengths of the two coupling wires 43a and 43b become substantially equal on the inside of bend and the outside of bend of the bend tube 21, and a difference in path length is small. Therefore, the coupling wires 43a and 43b keep the engagement state in which the protrusion portions 31 and reception portions 32 of the swivel contact portions are put in contact in the respective node rings 22, and the required bend operation of the bend tube 21 is secured. Moreover, the entire length of the coupling wire 43a, 43b is kept at a predetermined preset value and is invariable at the time of bending of the bend tube 21, and the bending operation of the bend tube 21 is not hindered. In particular, since there is no need to apply initial tension to the coupling wire 43a, 43b, the bend operation of the bend tube 21 can smoothly be performed.

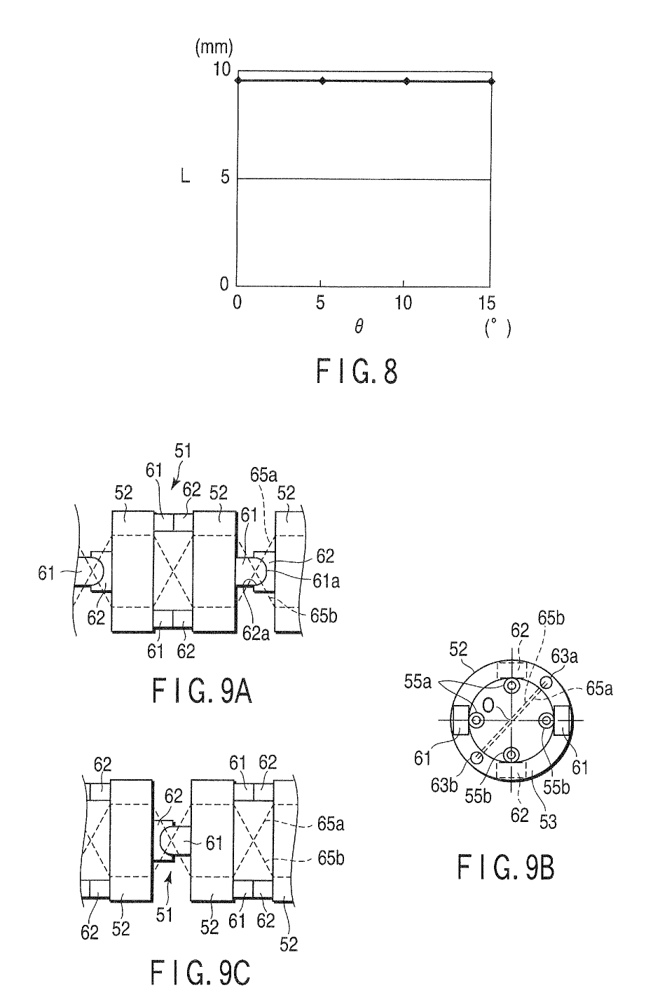

FIG. 7 shows a calculation result indicating that there is no difference in path length of the coupling wire 43a, 43b on the inside of bend and the outside of bend of the bend tube 21. FIG. 7 shows the variation of an inter-opening distance (L) of the coupling wire hold portions of the neighboring node rings 22 (i.e. the distance of the line segment AC or line segment DE), relative to the bend angle (θ) of the neighboring node rings 22. Specifically, FIG. 7 shows the difference (ΔL) between the inter-opening distance (L=L0) of the coupling wire hold portions in the case where the bend tube 21 is straight (i.e. the nor-bent state with the bend angle (θ) of 0°) and the inter-opening distance (L) of the coupling wire hold portions in the case where the bend tube 21 is bent (i.e. the bent state with the bend angle (θ) being not 0°), and the ratio (r) of this difference. FIG. 8 is a graph showing the relationship between the bend angle (θ) of the neighboring node rings 22 and the inter-opening distance (L) of the coupling wire hold portions. As is clear from this result, even if the bend tube 21 is bent, the variation in path length of the coupling wire 43a, 43b is within a practically negligible range, and the length of the coupling wire 43a, 43b can be kept at a predetermined value.

Although the results of FIG. 7 and FIG. 8 show the relationship between two neighboring node rings 22, the variation in path length of the coupling wire 43a, 43b is negligible even in the case where many node rings 22 are coupled. Even in the case of using a greater number of node rings 2, than usual (e.g. about 15 node rings 22), the engagement state of the swivel contact portions can be maintained by the coupling wires 43a and 43b, and this case is fully applicable. In particular, if the swivel contact portion is implemented by contact engagement between a projection portion and a recess portion, the invention is applicable to the case of using a still greater number of node rings 22.

According to the above-described structure, the contact engagement state of the swivel contact portions of the node rings 22 can be maintained in the state in which the coupling wires 43a and 43b are extended without initial tension. IF the path length of the coupling wire 43a, 43b is set at a predetermined length, even if the bend tube 21 is bent, there occurs no great difference in length of the coupling wire 43a, 43b, and the entire length of the coupling wire 43a, 43b is determined to be substantially constant. Therefore, the bending performance of the bend tube 21 can be maintained, without depending on the outer sheath structure. Furthermore, since the length of the coupling wire 43a, 43b can be kept in the predetermined state without the application of initial tension, the bending operation of the bend tube 21 is not hindered and the bending operation of the bend tube 21 can smoothly be performed.

FIG. 9A to FIG. 11 show a bend tube 51 of an endoscope according to a second embodiment of the present invention. This bend tube 51 is configured such that node rings 52 are arranged in line in the longitudinal direction of the bend section 15 (see FIG. 1), and the node rings 52 neighboring in the back-and-forth direction are coupled so as to be pivotable alternately in four directions, namely in upper and lower directions and in left and right directions.

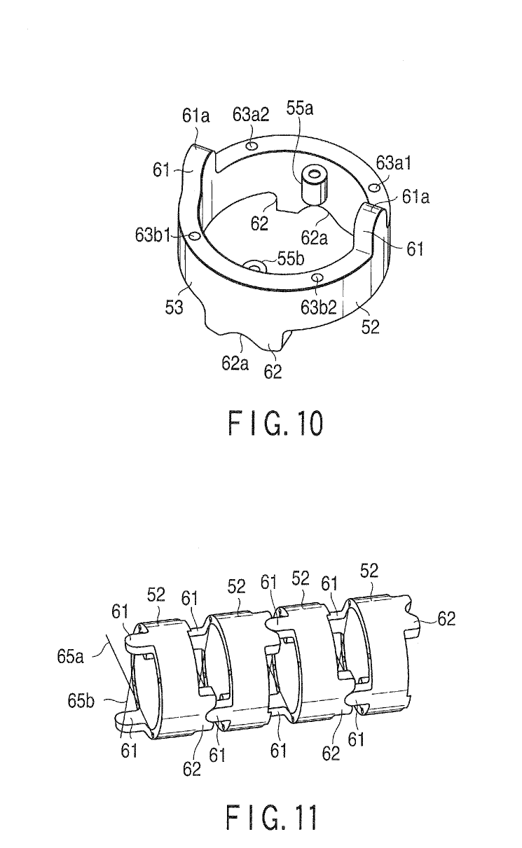

FIG. 10 shows a single-unit node ring 52. A pair of tongue-shaped protrusion portions 61, which are formed on one of end edges of the single-unit node ring 52, and a pair of tongue-shaped reception portion 62, which are provided on the other end edge of the same node ring 52, are disposed with a displacement of 90° about the center axis of the node ring 52. Specifically, the paired protrusion portions 61, which are disposed on one end edge of a node ring body 53, and the paired reception portions 62, which are disposed on the other end edge of the same node ring body 53, are disposed with a displacement of 90° about the center axis of the node ring 52. Thereby, as shown in FIG. 11, the protrusion portions 61 and reception portions 62, which are opposed between the neighboring node rings 52, are abutted upon each other. The projection portions 61 and reception portions 62 are formed integral with the node ring body 53.

Arcuate projection portions 61a are formed at projecting distal ends of the protrusion portions 61, and arcuate recess portions 62a are formed at projecting distal ends of the reception portions 62′. The projection portion 61a and the recess portion 62a are formed to have the same size (radius) of an arc. Accordingly, the projection portions 61a of the protrusion portions 61 of one node ring 52 are slidably fitted in the recess portions 62a of the reception portions 62 of the other node ring 52, and the projection portions 61a and the recess portions 62a are pivotably engaged. Thereby, vertically pivotable swivel contact portions and horizontally pivotable swivel contact portions are alternately provided between the coupled node rings 52. Therefore, the bend tube 51 can be bent in four directions, namely in upward, downward, leftward and rightward directions.

As shown in FIG. 9B, a pair of coupling wire hold holes 63a and 63b are formed between one end edge and the other end edge of the node ring body 53. The paired coupling wire hold holes 63a and 63b are disposed at point-symmetric positions with respect to the center axis (O) of the node ring 52, deviating from the protrusion portions 61 and reception portions 62 in the circumferential direction of the node ring 52. In addition, the coupling wire hold holes 63a and 63b are disposed at equidistant positions from the center axis (O) of the node ring body 53. An imaginary line passing through the paired coupling wire hold holes 63a and 63b passes through the center axis (O) of the node ring body 53. Specifically, the paired coupling wire hold holes 63a and 63b are obliquely disposed to be two-fold rotational-symmetric with respect to the center axis (O) of the node ring body 53. As shown in FIG. 9B, the paired coupling wire hold holes 63a and 63b are symmetrically disposed at oblique positions, with equal displacements of 45° in the circumferential direction of the bend tube 51 from the positions of the protrusion portion 61 and reception portion 62.

In general, it should suffice if only one pair of coupling wire hold holes 63a and 63b are provided in association with a pair of coupling wires which are assembled to the node rings 52, as described above. In the case where the bend section 15 is bent in the upward, downward, leftward and rightward directions, if such a configuration is adopted that each node ring 52 is provided with one pair of coupling wire hold holes 63a and 63b, the positions of the coupling wire hold holes 63a and 63b of each node ring 52 would be displaced when the node rings 52 are assembled. Thus, in the case of the configuration that each node ring 52 provided with one pair of coupling wire hold holes 63a and 63b, it is necessary to prepare two kinds of node rings 52 in which the positions of coupling wire hold holes are mirror-symmetric and are different.

On the other hand, in the present embodiment, as shown in FIG. 10, two kinds of pairs of coupling wire hold holes, which are mirror-symmetric, are formed in advance in the single-unit node ring 52. When the node rings 52 are assembled, one of the sets of coupling wire hold holes, which are arranged in line, is selected so that the coupling wiring hold holes may be disposed in line. Specifically, as shown in FIG. 10, one node ring 52 is provided with a set of paired coupling wire hold holes 63a1 and 63b1 (the coupling wire holes disposed at an upper right position and a lower left position) and another set of paired coupling wire hold holes 63a2 and 63b2 (the coupling wire holes disposed at an upper left position and a lower right position). The paired coupling wire hold holes 63a1 and 63b1 and the paired coupling wire hold holes 63a2 and 63b2 are disposed at oblique positions with equal displacements or 45° in the circumferential direction of the bend tube form the positions of the protrusion portions 61 and reception portions 62.

In the case where the node rings 52 each having the above-described structure are used and assembled, either the coupling wire hold holes 63a1 and 63b1 or the coupling wire hold holes 63a2 and 63b2 are arranged in line in parallel to the center axis (O) of the node rings 52. At this time, four juxtaposition sections of coupling wire hold holes are formed. One pair of juxtaposition sections may be selected from the four juxtaposition sections as the coupling wire hold holes through which the coupling wires are passed.

If the node rings 52 of the above-described double-use configuration are used, there is no need to separately prepare the node ring 52 of the kind in which the paired coupling wire hold holes 63a1 and 63b1 are provided, and the node ring 52 of the kind in which the paired coupling wire hold holes 63a2 and 63b2 are provided, and it should suffice if the node rings 52 of one kind are prepared.

As shown in FIG. 11, the node rings 52 are arranged in line, and the protrusion portions 61 and reception portions 62 are put in contact. In this case, the swivel contact portions of protrusion portions 61 and reception portions 62, which are positioned on the left and right sides, and the swivel contact portions of protrusion portions 61 and reception portions 62, which are positioned on the upper and lower sides, are alternately disposed. Accordingly, the bend tube 51 can be bent in four directions, namely upward, downward, leftward and rightward directions. For example, as shown in FIG. 9B, the coupling wire hold hole 63a at the upper right position and the coupling wire hold hole 63b at the lower left position are used as a set of paired coupling wire hold holes. Specifically, like the preceding embodiment, two coupling wires 65a and 65b can be disposed such that the coupling wires 65a and 65b are passed through the coupling wire hold holes 63a and 63b that are used.

In the node ring 52 of the present embodiment, as shown in FIG. 9B and FIG. 10, operation wire guide tubes 15a and 55b are formed integral with the node ring body 53 in a manner to project inward from the inner peripheral surface of the node ring body 53. The operation wire guide tubes 55a and 55b are disposed at positions corresponding to the positions of the reception portions 62.

In the present embodiment, too, the operation wire guide tubes 55a and 55b, the protrusion portions 61, the reception portions 62 and the coupling wire hold holes 63a and 63b are disposed to be point-symmetric about the center axis (O) of the node ring body 53, respectively. The coupling wire hold holes 63a and 63b are disposed at positions with displacements of, e.g. 45° in the circumferential direction of the bend tube 51, in a manner to avoid the operation wire guide tubes 55a and 55b, the protrusion portions 61 and the reception portions 62. Thus, the interference of the coupling wire hold holes 63a and 63b with other parts can be avoided as much as possible.

In this embodiment, like the above-described embodiment, the coupling wires 65a and 65b are individually passed through the different coupling wire hold holes 63a and 63b of the respective node rings 52. In this case, in the state in which the bend tube 51 is not bent and is straight (i.e. the non-bent state), the point, at which the line segment connecting the lead-out opening ends of the coupling wire hold holes 63a and 63b of the node rings 52 is halved, is set in such a relationship that this point is a point of intersection between the plane including the center axis (O) of the bend tube 51 and this line segment. At the same time, the point, at which this line segment is halved, intersects with the plane including the pivotal center axis of the swivel contact portions which pivotably couple the neighboring node rings 52. Therefore, the same operation as with the first embodiment can be performed.

The following advantageous effect can be obtained with the above-described structure. Specifically, the coupling wires 65a and 65b are passed through the coupling wire hold holes 63a and 63b of the respective node rings 52, with their insertion ends being alternated in a cross-coupling fashion. Therefore, by the same principle as in the above-described first embodiment, the coupling wires 65a and 65b secure the bend operation of the bend tube 51, while keeping the engagement state in which the protrusion portions 61 and reception portions 62 of the swivel contact portions are put in contact.

In the node ring 52 shown in FIG. 10, root only a pair of coupling wire hold holes 63a1 and 63b1 through which the paired coupling wires 65a and 65b are passed, but also another pair of coupling wire hold holes 63a2 and 63b2 are formed in advance. Therefore, it should suffice if the node rings 52 of one kind are prepared, without preparing two kinds of node rings with different positions of coupling wire hold holes which hold the paired coupling wires 65a and 65b, and the double-purpose use of components can be achieved. Besides, with use of the other set of coupling wire hold holes which were not used, coupling wires may also be passed through these coupling wire hold holes, with the insertion ends of the coupling wires being alternated in a cross-coupling fashion.

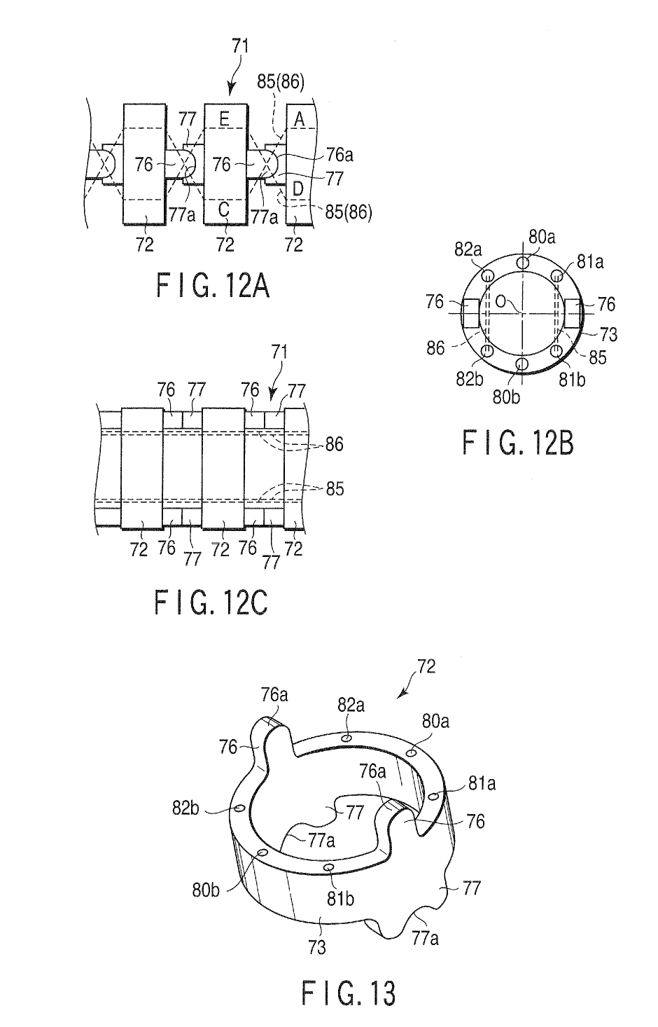

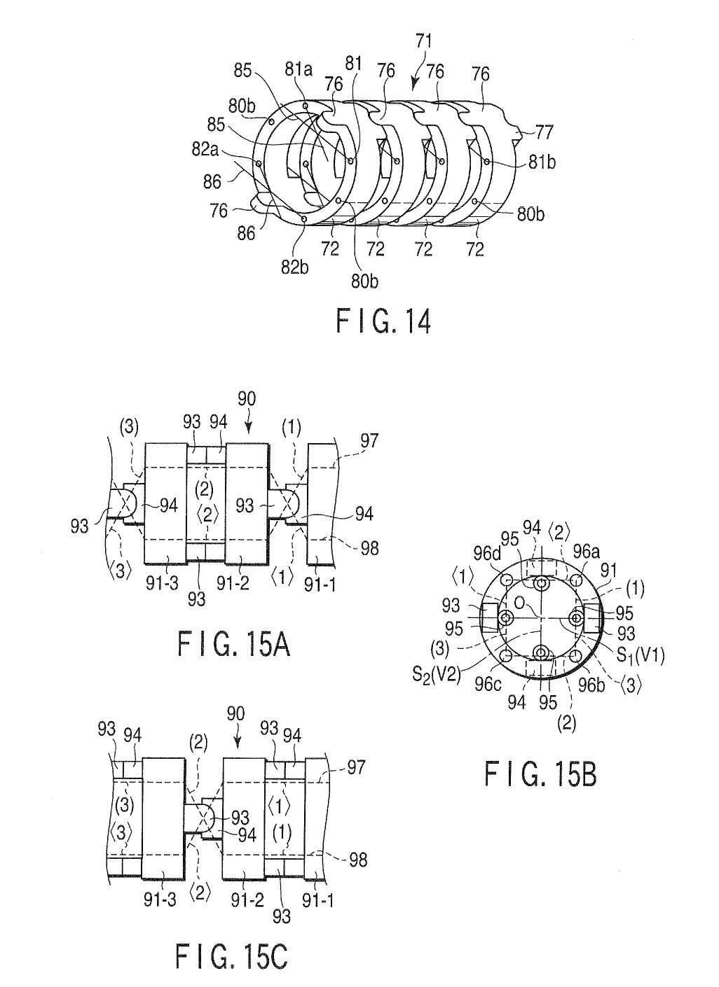

FIG. 12A to FIG. 14 show an endoscope bend tube 71 according to a third embodiment of the present invention. The endoscope bend tube 71 of this embodiment is configured such that node, rings 72, as shown in FIG. 13, are arranged in line, as shown in FIG. 12A, FIG. 12C and FIG. 14, and the neighboring node ring 72 are coupled in a manner to be pivotable in the up-and-down direction by swivel contact portions which are described later.

FIG. 13 shows a single-unit node ring 72. The node ring 72 includes a node ring body 73, a pair of tongue-shaped protrusion portions 76 which are formed on one of end edges of the node ring body 73, and a pair of tongue-shaped reception portions 77 which are provided on the other end edge of the node ring body 73. The paired tongue-shaped protrusion portions 76 are disposed on the left and right sides of the one end edge of the node ring body 73. Similarly, the paired tongue-shaped reception portions 77 are disposed on the left and right sides of the other end edge of the node ring body 73.

The protrusion portions 76 of one node ring 72 and the reception portions 77 of another neighboring node ring 77 are mutually put in contact, thereby constituting swivel contact portions which pivotably couple the neighboring node rings 72. Arcuate projection portions 76a are formed at projecting distal ends of the protrusion portions 76, and arcuate recess portions 77a are formed at projecting distal ends of the reception portions 77. The projection portion 76a and the recess portion 77a are formed to have the same size (radius) of an arc. Accordingly, the projection portions 76a of the protrusion portions 76 are closely fitted in, and slidably engaged with, the recess portions 77a of the reception portions 77. Thereby, swivel contact portions are constituted, which become hinge portions of the contact pair for coupling the neighboring front and rear node rings 72 in a manner to be pivotable about the center of the arc (the pivotal center axis).

As shown in FIG. 12B and FIG. 13, operation wire guide holes 80a and 80b are vertically symmetrically formed between one end face and the other end face of the node ring body 13. The operation wire guide holes 80a and 80b penetrate straight in the same direction as the direction of the center axis (O) of the node ring body 73. An imaginary line connecting the upper and lower operation wire guide holes 80a and 80b passes through the center axis (O) of the node ring body 73 and intersects at right angles with the center axis (O). In addition, an imaginary line connecting the centers of the arc of the paired left and right protrusion portions 76 and an imaginary line connecting the centers of the arc of the left and right reception portions 77 pass through the center axis (O) of the node ring body 73 and intersect at right angles with the center axis (O). The structure, which has been described thus far, is the same as that of the node ring 22 of the first embodiment shown in FIG. 2.

Further, in the present embodiment, four coupling wire hold holes 81a, 81b, 82a and 82b, which function as coupling wire hold portions, are provided between one end face and the other end face of the node ring body 73. In this embodiment, a set of first coupling wire hold holes 81a and 81b, which is a vertically disposed pair, and a set of second coupling wire hold holes 82a and 82b, which is a vertically disposed pair, are formed. These two sets of coupling wire hold holes 81a, 81b, 82a and 82b are formed to penetrate straight in the same direction as the direction of the center axis (O) of the node ring body 73.

As shown in FIG. 12B, the set of first coupling wire hold holes 81a and 81b is disposed on the right side of an imaginary line which connects the upper and lower operation wire guide holes 80a and 80b. The set of second coupling wire hold holes 82a and 82b is disposed on the left side of the imaginary line which connects the upper and lower operation wire guide holes 80a and 80b. In other words, the set of first coupling wire hold holes 81a and 81b and the set of second coupling wire hold holes 82a and 82b are disposed symmetric in the right-and-left direction with respect to the imaginary line which connects the upper and lower operation wire guide holes 80a and 80b.

The four coupling wire hold holes 81a, 81b, 82a and 82b are disposed at oblique positions along the circumferential direction of the node ring body 73 so as to avoid interference with the positions of the upper and lower operation wire guide holes 80a and 80b and the positions of the protrusion portions 76 and reception portions 77 which constitute the swivel contact portions. In this embodiment, the four coupling wire hold holes 81a, 81b, 82a and 82b are disposed at positions of 45° in the circumferential direction of the node ring body 73 from the positions of the guide holes 80a and 80b, protrusion portions 76 and reception portions 77, respectively. Furthermore, the coupling wire hold holes 81a, 81b, 82a and 82b are disposed at equidistant positions from the center axis (O) of the node ring body 73. In short, the first coupling wire hold holes 81a and 81b and second coupling wire hold holes 82a and 82b are disposed to be four-fold rotational-symmetric with respect to the center axis (O) of the node ring body 73.

An imaginary line connecting the right-side set of first coupling wire hold holes 81a and 81b and an imaginary line connecting the left-side set of second coupling wire hold holes 82a and 82b are halved by an imaginary line connecting the centers of the arc of the paired left and right protrusion portions 76 (and an imaginary line connecting the centers of the arc of the paired left and right reception portions 77). In addition, the right-side set of first coupling wire hold holes 81a and 81b and the left-side set of second coupling wire hold holes 82a and 82b are disposed symmetric in the right-and-left direction with respect to a vertical line extending through the center axis (O) of the node ring body 73.

As described above, the set of first coupling wire hold holes 81a and 81b and the set of second coupling wire hold holes 82a and 82b are disposed at oblique positions on the left and right sides or at oblique positions on the upper and lower sides with respect to the center axis (O) of the node ring body 73. Thus, the first coupling wire hold holes 81a and 81b and second coupling wire hold holes 82a and 82b are disposed to be also symmetric in the oblique directions. The four coupling wire hold holes 81a, 81b, 82a and 82b are disposed in a relationship of four-fold rotational symmetry with respect to the center axis (O) of the node ring body 73. The operation wire guide holes 80a and 80b, the protrusion portions 76, the reception portions 77 and the coupling wire hold holes 81a, 81b, 82a and 82b are disposed at substantially equidistant positions from the center axis (O) of the node ring body 73, and are disposed at equal intervals of 45° in the circumferential direction.

In the case where the plural node rings 72 are coupled by two coupling wires 85 and two coupling wires 86, as shown in FIG. 12A to FIG. 12C, a set of two coupling wires 85 and another set of two coupling wires 86 are passed through the set of first coupling wire hold holes 81a and 81b and the set of second coupling wire hold holes 82a and 82b. For example, the two coupling wires 85, which are assembled to the first coupling wire hold holes 81a and 81b, are passed through the coupling wire hold holes 81a and 81b which are positioned on the upper and lower sides of each node ring 72. Then, the coupling wires 85 are individually inserted into the coupling wire hold holes 81a, 81b by alternating the upper-side and lower-side coupling wire hold holes 81a and 81b of every other node ring 72. Accordingly, the two coupling wires 85 cross in obliquely upward and downward directions between the node rings 72, and the two coupling wires 85 are passed between the neighboring node rings 72 in a cross-coupling fashion.

Similarly, the two coupling wires 86, which are assembled to the second coupling wire hold holes 82a and 82b, are passed through the coupling wire hold holes 82a and 82b which are positioned on the upper and lower sides of each node ring 72. Then, the coupling wires 86 are individually inserted into the coupling wire hold holes 82a, 82b by alternating the upper-side and lower-side coupling wire hold holes 82a and 82b of every other node ring 72. Accordingly, the two coupling wires 86 cross in obliquely upward and downward directions between the node rings 72, and the two coupling wires 86 are passed between the neighboring node rings 72 in a cross-coupling fashion.

As described above, the coupling wires 85, 86 are alternately passed through the two coupling wire hold holes at different positions in the up-and-down direction between the node rings 72 which neighbor in the back-and-forth direction. For example, the coupling wire 85, which has been passed through the coupling wire hold hole 81a at the upper-side position of the rear-side node ring 72, is led out of the front-side opening (lead-out end) of the coupling wire hold hole 81a, and then extends obliquely toward the coupling wire hold hole 81b at the lower-side position of the neighboring front-side node ring 72. The coupling wire 85 is then inserted into the coupling wire hold hole 81b of the node ring 72 from the rear-side opening (lead-out end) of the coupling wire hold hole 81b of the node ring 72. The other coupling wire 85 extends obliquely from the front-side opening of the coupling wire hold hole 81b at the lower-side position of the rear-side node ring 72 toward the coupling wire hold hole 81a at the upper-side position of the neighboring front-side node ring 72. Then, between the node rings 72 which neighbor in the back-and-forth direction, the two coupling wires 85 are disposed in an obliquely crossing configuration. The coupling wires 86 are similarly passed through the other set of second coupling wire hold holes 82a and 82b and are assembled to the node rings 72.

In the non-bent state in which the bend tube 71 is not bent, a plane including the center axis (O) of the bend tube 71 and the imaginary line connecting the centers of the arc of the paired left and right protrusion portions 76 (or an imaginary line connecting the centers of the arc of the paired left and right reception portions 77) is disposed in such a relationship as to halve the line segment “AC” connecting the first coupling wire hold holes 81a and 81b of the neighboring node rings 72 and the line segment “DE” connecting the second coupling wire hold holes 82a and 82b of the neighboring node rings 72. Therefore, by the same principle as in the first embodiment, even if the bend tube 71 is bent, the lengths of the coupling wires 85 and 86 are not substantially varied, and the coupling wires 85 and 86 maintain the engagement state in which the swivel contact portions of the node rings 72 are put in contact, and secure the bend operation of the bend tube 71.

The following advantageous effect can be obtained by the above-described structure. In the present embodiment, the two sets of coupling wires 85 and 86 are provided in association with the set of first coupling wire hold holes 81a an 81b and the set of second coupling hold holes 82a and 82b, and the respective sets of coupling wires 85 and 86 are vertically disposed on the right and left regions of the bend tube 71. Therefore, an empty central part can be created in the bend tube 71. According to the arrangement of the coupling wires 85 and 86 of the present embodiment, built-in components can easily be disposed within the bend tube 11.

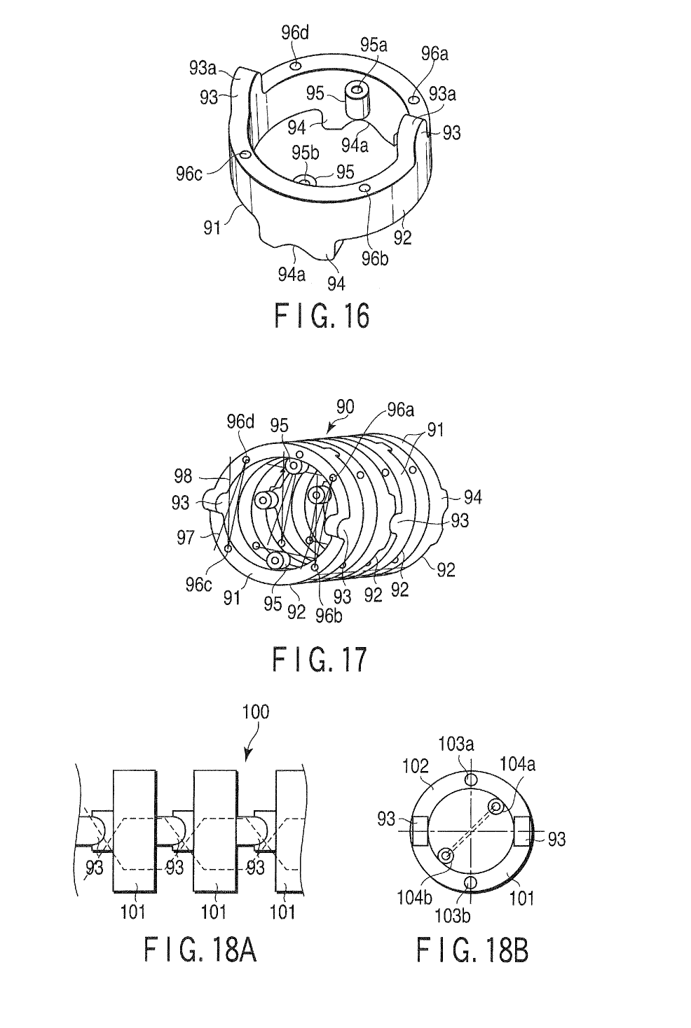

FIG. 15A to FIG. 17 show an endoscope bend tube 90 according to a fourth embodiment of the present invention. As shown in FIG. 15A, FIG. 15C and FIG. 17, in the endoscope bend tube 90, a plurality of node rings 91 are arranged in line, and a pair of neighboring node rings 91 are coupled by swivel contact portions (to be described later) so as to be pivotable in the up-and-down direction or in the right-and-left direction. Thereby, in the present embodiment, the bend tube 90, as a whole, is configured to be bendable in the up-and-down direction and in the right-and-left direction.

FIG. 16 shows a single-unit node ring 91. The node ring 91 includes a node ring body 92, a pair of tongue-shaped protrusion portions 93, which are formed on one of end edges of the node ring body 92, and a pair of tongue-shaped reception portion 94, which are provided on the other end edge of the same node ring body 92. In the same node ring 91, the paired protrusion portions 93 and the paired reception portions 94 are disposed with a displacement of 90° in the circumferential direction of the node ring 91. For example, in the case where the paired protrusion portions 93 are disposed at the left and right positions, the paired reception portions 94 are disposed at the upper and lower positions. In addition, the paired protrusion portions 93 and the paired reception portions 94 of the same node ring 91 are disposed in a symmetric relationship with a displacement of 90° in the circumferential direction of the node ring 91.

Like the above-described first embodiment, arcuate projection portions 93a are formed at projecting distal ends of the protrusion portions 93, and arcuate recess portions 94a are formed at projecting distal ends of the reception portions 94. The projection portion 93a and the recess portion 94a have the same size (radius) of an arc. Accordingly, the projection portions 93a of the protrusion portions 93 of the node ring 91 are closely fitted in, and slidably engaged with, the recess portions 94a of the reception portions 94 of the neighboring node ring 91.

The protrusion portions 93 and reception portions 94 of the neighboring node rings 91 couple the neighboring node rings 91 in a manner to be pivotable about the centers of the arc, thereby constituting swivel contact portions which become hinge portions of the contact pair. The pivotal axis of the swivel contact portion is displaced by 90° about the center axis (O) of the node ring successively from node ring 91 to node ring 91. Accordingly, the direction of pivotal movement of each neighboring node ring 91 is changed alternately between the up-and-down direction and the right-and-left direction. As a result, the bend tube 90, as a whole, is bendable in the up-and-down direction and right-and-left direction.

As shown in FIG. 16, in the node ring body 92, a pair of guide tubes 95 are formed in a manner to project inward from the inner peripheral surface of the node ring body 92. The paired guide tubes 95 form operation wire guide holes 95a and 95b which penetrate straight in the same direction as the direction of the center axis (O) of the node ring body 92. The guide tubes 95 are disposed at positions corresponding to the reception portions 94. Alternatively, the guide tubes 95 may be disposed at positions corresponding to the protrusion portions 93, or at positions corresponding to both the protrusion portions 93 and reception portions 94. Shown here is the example in which the guide tubes 95 are disposed only at positions corresponding to the reception portions 94.

The paired protrusion portions 93 are formed on one of end edges of the node ring body 92 of each node ring 91, and the paired reception portion 94 are formed on the other end edge of the node ring body 92. Thereby, as shown in FIGS. 15A to 15C and FIG. 17, the node rings 91 are assembled in the state in which the contact positions of the protrusion portions 93 and reception portions 94 of the neighboring node rings 91 are displaced by 90° about the center axis (O). At this time, the protrusion portions 93 and reception portions 94 of the neighboring node rings 91 are put in contact, thereby constituting the swivel contact portions. The swivel contact portions are alternately disposed with a displacement of 90° in the circumferential direction of the node ring 91.

As shown in FIG. 15B, in the state in which the plural node rings 91L are assembled, an imaginary line V1, which connects the centers of the paired operation wire guide holes 95a and 95b that are disposed in the up-and-down direction, and an imaginary line V2, which connects the rotational centers of the paired upper and lower swivel contact portions, pass through the center axis (O) of the node ring body 92 and intersect at right angles with the center axis (O). In addition, an imaginary line V₁, which connects the centers of the paired operation wire guide holes 95a and 95b that are disposed in the right-and-left direction, and an imaginary line V1, which connects the rotational centers of the paired left and right swivel contact portions, pass through the center axis (O) of the node ring body 92 and intersect at right angles with the center axis (O).

Operation wires (not shown) are individually passed through the upper and lower operation wire guide holes 95a and 95h, and the upper and lower operation wires are pushed and pulled. Thereby, the node ring 91 can be pivoted in the up-and-down direction about the pivotal centers of the paired swivel contact portions which are disposed on the left and right sides. In addition, left and right operation wires (not shown) are individually passed through the left and right operation wire guide holes 95a and 95b, and the left and right operation wires are pushed and pulled. Thereby, the node ring 91 can be pivoted in the right-and-left direction about the pivotal centers of the paired swivel contact portions which are disposed on the upper and lower sides.

As shown in FIG. 15B and FIG. 16, the node ring body 92 is provided with four coupling wire hold holes 96a, 96b, 96c and 96d, which function as coupling wire hold portions. Each of the four coupling wire hold holes extends in parallel to the center axis (O) of the node ring body 92, and is formed to penetrate straight between both end faces of the node ring body 92.

The positions of the four coupling wire hold holes 96a, 96b, 96c and 96d are set so as to avoid the swivel contact portions which are positioned on the upper, lower, left and right sides. For example, as shown in FIG. 15B, the respective coupling wire hold holes 96a, 96b, 96c and 96d are disposed at oblique positions with displacements of 45° in the circumferential direction of the node ring body 92 from the positions of the upper, lower, left and right swivel contact portions.

The four coupling wire hold holes 96a, 96b, 96c and 96d are disposed at equidistance positions from the imaginary line V1 connecting the pivotal centers of the upper and lower swivel contact portions and from the imaginary line V2 connecting the pivotal centers of the left and right swivel contact portions, and are also disposed at equidistance positions from the center axis (O) of the bend tube 90. The four coupling wire hold holes 96a, 96b, 96c and 96d are disposed on the node ring body 92 at positions of four-fold rotational symmetry about the axis of the node ring body 92.

Each of an imaginary line connecting the centers of the coupling wire hold holes 96a and 96b, which are disposed at corresponding upper and lower positions, and an imaginary line connecting the centers of the coupling wire hold holes 96c and 96d, which are also disposed at corresponding upper and lower positions, is halved by the imaginary line V2 connecting the pivotal centers of the left and right swivel contact portions, is parallel to the imaginary line V1 connecting the pivotal centers of the upper and lower swivel contact portions, and intersects at right angles with the imaginary line V2 connecting the pivotal centers of the left and right swivel contact portions (see FIG. 15B).

In addition, each of an imaginary line connecting the centers of the coupling wire hold holes 96a and 96d, which are disposed at corresponding right and left positions, and an imaginary line connecting the centers of the coupling wire hold holes 96b and 96c, which are also disposed at corresponding right and left positions, is halved by the imaginary line V1 connecting the pivotal centers of the upper and lower swivel contact portions, is parallel to the imaginary line V2 connecting the pivotal centers of the left and right swivel contact portions, and intersects at right angles with the imaginary line V1 connecting the pivotal centers of the upper and lower swivel contact portions (see FIG. 15B).

Next, referring to FIGS. 15A, 15B and 15C, a description is given of the assembly state of coupling wires 97 and 98 which couple the node rings 91. In this embodiment, at least two coupling wires 97 and 98 are provided in order to balance the coupling force of the node rings 91, and these two coupling wires 97 and 98 are used. The first coupling wire 97 and second coupling wire 98 are successively passed from the coupling wire hold holes of a rear-side node ring 91 to the coupling wire hold holes of a front-side node ring 91. For example, one of the four coupling wire hold holes 96a, 96b, 9 c and 98d of each node ring 91 is successively selected counterclockwise from node ring 91 to node ring 91, and the coupling wire 97, 98 is successively passed into these coupling wire hold holes.

Specifically, a portion (1) of the first coupling wire 97 is led out of the coupling wire hold hole 96a, which is positioned on the upper right side in FIG. 15B, in a first node ring 91-1 which is positioned on the rightmost (rearmost) side in FIG. 15A and FIG. 15C. The portion (1) of the first coupling wire 97 extends beyond a plane S1 including the imaginary line (pivotal center axis) which connects the pivotal centers of the left and right swivel contact portions which pivot the first node ring 91-1 and a neighboring second node ring 91-2, is led to the opposite side (lower side) of the plane S1, and is inserted into the coupling wire hold hole 96b of the front-side neighboring second node ring 91-2.

The first coupling wire 97, which has been inserted into the coupling wire hold hole 96b of the second node ring 91-2, is led out forward from the other end side opening (lead-out end) of the coupling wire hold hole 96b. This led-out portion (2) extends beyond a plane S2 including the imaginary line (pivotal center axis) which connects the pivotal centers of the upper and lower swivel contact portions between the second node ring 91-2 and a front-side neighboring third node ring 91-3, is led to the opposite side of the plane S2, and is inserted into the coupling wire hold hole 96c of the neighboring third node ring 91-3 that is positioned on the lead-out side.

The first coupling wire 97, which has been inserted into the coupling wire hold hole 96c of the third node ring 91-3, is led out forward from the other end side opening (lead-out end) of the coupling wire hold hole 96c of the third node ring 91-3. This led-out portion (3) extends beyond the plane S1 including the imaginary line (pivotal center axis) which connects the pivotal centers of the left and right swivel contact portions between the third node ring 91-3 and a front-side neighboring fourth node ring 91-4, is led to the opposite side of the plane S1, and is inserted into the coupling wire hold hole 96d of the neighboring fourth node ring 91-4.

Further, the first coupling wire 97, which has been inserted into the coupling wire hold hole 95d of the fourth node ring 91-4, is led out from the other end side opening (lead-out end) of the coupling wire hold hole 96d. This led-out portion extends beyond the plane S2 including the imaginary line (pivotal center axis) which connects the pivotal centers of the upper and lower swivel contact portions between the fourth node ring 91-4 and a further front-side neighboring node ring 91, is led to the opposite side of the plane S2, and is inserted into the coupling wire hold hole 96a of the neighboring node ring 91 that is positioned on the lead-out side.

In this manner, the first coupling wire 97 is passed successively through the coupling wire hold holes 96a, 96b, 96c and 96d of the respective node rings 91.

On the other hand, in like manner, the second coupling wire 98 is passed successively through the coupling wire hold holes 96a, 96b, 96c and 96d of the respective node rings 91. The second coupling wire 98 is designed to be passed into the coupling wire hold holes of the respective node rings 91, with the order of passing being shifted by two coupling wire hold holes, compared to the case of the first coupling wire 97.

Specifically, the second coupling wire 98 is led out of the coupling wire hold hole 96c in the first node ring 91-1. A portion <1> of the second coupling wire 98, which has been led out of the coupling wire hold hole 96c of the first node ring 91-1, extends beyond the plane S1 including the imaginary line (pivotal center axis) which connects the pivotal centers of the left and right swivel contact portions which pivot the first node ring 91-1 and the front-side neighboring second node ring 91-2, is led to the opposite side (upper side), and is inserted into the coupling wire hold hole 96d of the second node ring 91-2.

A portion <2> of the second coupling wire 98, which has been inserted into the coupling wire hold hole 96d of the second node ring 91-2, is led out from the other end side opening (lead-out end) of the coupling wire hold hole 96d. This led-out, portion <2> extends beyond the plane S2 including the imaginary line (pivotal center axis) which connects the pivotal centers of the upper and lower swivel contact portions between the second node ring 91-2 and the front-side neighboring third node ring 91-3, is led to the opposite side (right side) of the plane S2, and is inserted into the coupling wire hold hole 96a of the neighboring third node ring 91-3.

Further, a portion <3> of the second coupling wire 98, which has been inserted into the coupling wire hold hole 96a of the third node ring 91-3, extends beyond the plane S1 including the imaginary line (pivotal center axis) which connects the pivotal centers of the left and right swivel contact portions between the third node ring 91-3 and the front-side neighboring fourth node ring 91-4, is led to the opposite side of the plane S1, and is inserted into the coupling wire hold hole 96b of the fourth node ring 91-4. In this manner, the second coupling wire 98 is passed successively through the coupling wire hold holes 96c, 96d, 96a and 96b of the respective node rings 91.

Thus, between the node rings 91, the second coupling wire 98 extends beyond the plane S1, S2 from the side opposite to the side from which the first coupling wire 97 extends, and the second coupling wire 98 is disposed between the node rings 91. Accordingly, as shown in FIG. 15A and FIG. 15C, between the neighboring node rings 91, the first coupling wire 97 and second coupling wire 98 are neither parallel nor crossing directly, but they cross in a skew fashion. Specifically, each of the first coupling wire 97 and second coupling wire 98 passes through the coupling wire hold holes 96a, 96b, 96c and 96d of the node rings 91 in a helical fashion about the center axis (O) of the bend tube 90, and is disposed along the periphery of the bend tube 90. The first coupling wire 97 and second coupling wire 98 cross in a skew fashion between the neighboring node rings 91. Although two coupling wires are used in this description, other coupling wires may similarly be passed by using the other two coupling wire hold holes which remain non-used.

The following advantageous effects can be obtained with the above-described structure. Specifically, in this embodiment, too, in the non-bent state in which the bend tube 90 is not bent, the line segment connecting the coupling wire hold holes 96a, 96b, 96c, 96d, through which the coupling wires 97, 98 are passed, is halved by the plane including the imaginary line (pivotal center axis) connecting the centers of the swivel contact portions of the node ring 91. Therefore, the same advantageous effect as in the first embodiment can be obtained. That is, the coupling wires 97 and 98 maintain the engagement state in which the swivel contact portions of the node rings 91 are put in contact, and secure the bend operation of the bend tube 91. In addition, each coupling wire 97, 98 is successively passed between the node rings 91 in a helical fashion, and passes through the peripheral part of the bend tube 90. Thus, since the coupling wire 97, 98 does not occupy the central part of the bend tube 90, a large space for installation of built-in components can be secured near the central part of the bend tube 90.

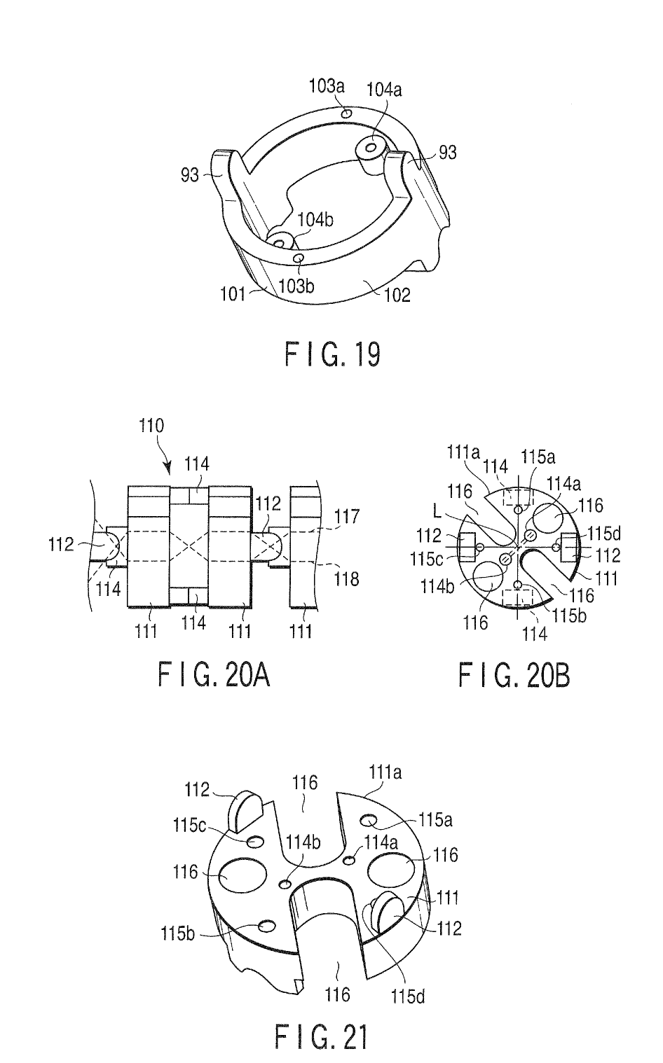

FIG. 18A, FIG. 18B and FIG. 19 show an endoscope bend tube 100 according to a fifth embodiment of the present invention. FIG. 19 shows a single-unit node ring 101. Like the node ring 22 of the first embodiment, two operation wire guide holes 103a and 103b are formed between one end edge and the other end edge of a node ring body 102. Coupling wire hold portions are formed by holes in two guide tubes 104a and 104b which project inward from the inner peripheral surface of the node ring body 102.

FIG. 20A, FIG. 20B and FIG. 21 show an endoscope bend tube according to a sixth embodiment of the present invention. In the endoscope bend tube 110 of this embodiment, a node ring 111 is formed not in a tubular shape, but a disc-shaped node ring body 111a is formed. Coupling wire hold holes 114a and 114b, which function as coupling wire hold portions, and operation wire guide holes 115a, 115b, 115c and 115d are formed by boring between one end edge and the other end edge of the disc-shaped node ring 111. Further, a plurality of space portions 116, in which other built-in components are disposed, are formed by cutting in the outer peripheral part of the node ring 111. Besides, in the node ring 111 of this embodiment, as shown in FIG. 20B, the coupling wire hold holes 114a and 114b are provided near the center axis (O) of the node ring 111.

In the present embodiment, since the coupling wire hold holes 114a and 114b can be disposed near the center of the node ring 111, coupling wires 117 and 118 can be disposed near the center of the node ring 111. Like the above-described first embodiment, the coupling wires 117 and 118 are disposed such that they are passed through the coupling wire hold holes 114a and 114b of the node rings 111 in a cross-coupling fashion.

In each of the above-described embodiments, the swivel contact portion is configured such that an arcuate projection portion is formed at a projecting distal end of the protrusion portion, and an arcuate recess portion is formed at a projecting distal end of the reception portion. The projection portion and the recess portion are fitted and engaged, thereby forming the contact portion. However, it should suffice if the swivel contact portion permits pivotal movement of neighboring node rings which arc put in contact. Thus, the swivel contact portion may not necessarily have the is relationship of the projection portion and recess portion. In addition, in each of the above-described embodiments, protrusion portions are formed on one end edge of the single-unit node ring and reception portions are formed on the other end edge thereof, thereby constituting the swivel contact portion on the node ring. However, use may be made of a bend tube including such a type of node ring that protrusion portions are provided on both end edges of the node ring, and such a type of node ring that reception portions are provided on both end edges of the node ring. Both types of node rings are alternately arranged, and the protrusion portions and reception portions are put in contact, thereby constituting swivel contact portions which pivotably couple a pair of node rings which neighbor in the back-and-forth direction.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope bend tube including a plurality of node rings which are arranged in line, wherein contact portions at which neighboring ones of the node rings are put in contact are provided between the neighboring node rings, and the plurality of node rings are coupled to be pivotable about pivotal center axes which correspond to pivotal centers of the contact portions, the endoscope bend tube comprising:
an operation wire which is disposed in the bend tube and bends the bend tube;
a coupling wire which is disposed between the node rings and couples the node rings; and
a coupling wire hold portion which is provided in the node ring and holds the coupling wire,
wherein in a case where the endoscope bend tube is in a non-bent state, lead-out ends of the coupling wire hold portions, which are opposed between a pair of the neighboring node rings, are disposed at such positions that a line segment connecting the lead-out ends is halved by a plane including a center axis in a longitudinal direction of the bend tube and a pivotal center axis of the pair of the node rings.

2. The endoscope bend tube according to claim 1, wherein the lead-out end of the coupling wire hold portion of one of the paired node rings and the lead-out end of the coupling wire hold portion of the other node ring that neighbors and is opposed to the one of the paired node rings are disposed at positions of two-fold rotational symmetry about the center axis in the longitudinal direction of the bend tube.

3. The endoscope bend tube according to claim 1, wherein the lead-out end of the coupling wire hold portion of one of the paired node rings and the lead-out end of the coupling wire hold portion of the other node ring that is opposed to the one of the paired node rings are disposed at positions of four-fold rotational symmetry about the center axis in the longitudinal direction of the bend tube.

4. The endoscope bend tube according to claim 1, wherein the coupling wire is disposed in a path passing through the line segment.

5. The endoscope bend tube according to claim 1, wherein each of the node rings includes at least two the coupling wire hold portions, and the node rings are coupled by two the coupling wires which are passed through the coupling wire hold portions of each node ring.

6. The endoscope bend tube according to claim 1, wherein each of the node rings includes at least four the coupling wire hold portions, and the node rings are coupled by four the coupling wires which are passed through the coupling wire hold portions of each node ring.

7. The endoscope bend tube according to claim 1, wherein the node ring includes an operation wire hold portion which holds the operation wire which bends the endoscope bend tube, and the coupling wire hold portion is disposed between the operation wire hold portion and a position corresponding to a part where the contact portion at which the neighboring node rings are put in contact is provided.

8. The endoscope bend tube according to claim 1, wherein the endoscope bend tube includes two the operation wires, and the endoscope bend tube is bendable in two directions by the two operation wires.

9. The endoscope bend tube according to claim 1, wherein the endoscope bend tube Includes four the operation wires, and the endoscope bend tube is bendable in four directions by the four operation wires.

10. The endoscope bend tube according to claim 1, wherein the coupling wire is a metallic stranded wire.

11. The endoscope bend tube according to claim 1, wherein the coupling wire is a resin twisted thread.

12. An endoscope including an endoscope bend tube which includes a plurality of node rings which are arranged in line, wherein contact portions at which neighboring ones of the node rings are put in contact are provided between the neighboring node rings, and the plurality of node rings are coupled to be pivotable about pivotal center axes which correspond to pivotal centers of the contact portions, the endoscope comprising:
an operation wire which is disposed in the bend tube and bends the bend tube;
a coupling wire which is disposed between the node rings and couples the node rings; and
a coupling wire hold portion which is provided in the node ring and holds the coupling wire,
wherein in a case where the endoscope bend tube is in a non-bent state, lead-out ends of the coupling wire hold portions, which are opposed between a pair of the neighboring node rings, are disposed at such positions that a line segment connecting the lead-out ends is halved by a plane including a center axis in a longitudinal direction of the bend tube and a pivotal center axis of the pair of the node rings.
